(19) United States
(12) Patent Application Publication
(10) Pub. No.: US 2012/0010569 A1
(43) Pub. Date: Jan. 12, 2012

(54) ACCESS DEVICE WITH TWIST LOCKING REMOVABLE CAP

(76) Inventor: Shailendra K. Parihar, Mason, OH (US)

(21) Appl. No.: 12/832,129

(22) Filed: Jul. 8, 2010

(51) Int. Cl. A61B 17/34

(52) U.S. Cl. 604/167.01

(57) ABSTRACT

A surgical access device comprises a cannula having a proximal end and a distal end. A housing is connected to the proximal end of the cannula and contains a zero-closure seal. The housing may also include a housing cap with a plurality of oblong apertures. A removable cap contains an instrument seal and comprises a plurality of distally extending L-shaped projections having a twist-to-lock engagement with the oblong apertures to attach the removable cap to the housing.

### BACKGROUND

The present invention relates in general to surgical devices and procedures, and more particularly to minimally invasive surgery.

Surgical procedures are often used to treat and cure a wide range of diseases, conditions, and injuries. Surgery often requires access to internal tissue through open surgical procedures or endoscopic surgical procedures. The term “endoscopic” refers to all types of minimally invasive surgical procedures including laparoscopic, arthroscopic, natural orifice intraluminal, and natural orifice transluminal procedures. Endoscopic surgery has numerous advantages compared to traditional open surgical procedures, including reduced trauma, faster recovery, reduced risk of infection, and reduced scarring. Endoscopic surgery is often performed with an insufflatory fluid present within the body cavity, such as carbon dioxide or saline, to provide adequate space to perform the intended surgical procedures. The insufflated cavity is generally under pressure and is sometimes referred to as being in a state of pneumoperitoneum. Surgical access devices are often used to facilitate surgical manipulation of internal tissue while maintaining pneumoperitoneum. For example, trocars are often used to provide a port through which endoscopic surgical instruments are passed. Trocars generally have an instrument seal, which prevents the insufflatory fluid from escaping while an instrument is positioned in the trocar.

While surgical access devices are known, no one has previously made or used a surgical access device in accordance with the present invention.

### SUMMARY

In one embodiment, a trocar may comprise a cannula having a proximal end and a distal end. A housing may be connected to the proximal end of the cannula, the housing containing a zero-closure seal. A removable cap may contain an instrument seal and comprise a plurality of distal projections providing a twist-to-lock attachment to the housing. The trocar may optionally include an obtruator. The trocar may include a housing cap with a seal engaging the removable cap. The housing cap may include a plurality of apertures dimensioned to receive the distal projections. The apertures may be oblong and oriented circumferentially.

The distal projections may each comprise a circumferentially oriented tab, which may optionally be oriented counter clock-wise. The distal projections and tabs may be generally L-shaped. The distal projections may alternatively comprise a laterally oriented tab. The housing cap may comprise a plurality of proximal projections each comprising a medially oriented tab adapted to mate with the laterally oriented tabs.

In another embodiment, a removable cap comprises an upper housing having a proximal face, a distal face, and a bore extending between the proximal and distal faces. An instrument seal may be positioned in the bore. A plurality of projections from the distal face may be adapted mate with a lower portion of an access device such that rotation of the upper housing about the bore in a first direction locks the upper housing to the lower portion, and rotation of the upper housing about the bore in a second direction unlock the upper housing from the lower portion.

The distal projections each comprise a circumferentially oriented tab. The tabs may be oriented counter clock-wise. The distal projections may each comprise a laterally oriented tab. Optionally, a raised ring on the distal face may circumscribe the bore and engage a seal on the lower portion.

The removable cap may be bundled with a trocar comprising a lower housing with a zero-closure seal, and a cannula extending distally from the lower housing, and may further comprise an obtruator.

In another embodiment, a surgical access device comprises a cannula having a proximal end and a distal end. A housing is connected to the proximal end of the cannula and contains a zero-closure seal. The housing may also include a housing cap with a plurality of oblong apertures. A removable cap contains an instrument seal and comprises a plurality of distally extending L-shaped projections having a twist-to-lock engagement with the oblong apertures to attach the removable cap to the housing.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are not necessarily drawn to scale, but rather to illustrate the principles of the invention.

FIG. 1 depicts a perspective view of an access device with a removable cap;

FIG. 2 depicts a perspective view of the access device of FIG. 1;

FIG. 3 depicts a perspective view of the access device of FIG. 1 showing a detailed view of an attachment mechanism for the removable cap;

FIG. 4 depicts an exploded view of the access device of FIG. 1;

FIG. 5 depicts an exploded view of a removable cap of FIG. 1;

FIG. 6 depicts a detailed view of a housing cap and seal of FIG. 1;

FIG. 7 depicts a detailed view of an alternative housing cap and seal;

FIG. 8 depicts an alternative attachment mechanism for a removable cap;

FIG. 9 depicts an alternative attachment mechanism for a removable cap; and

FIG. 10 depicts an alternative attachment mechanism for a removable cap.

### DETAILED DESCRIPTION

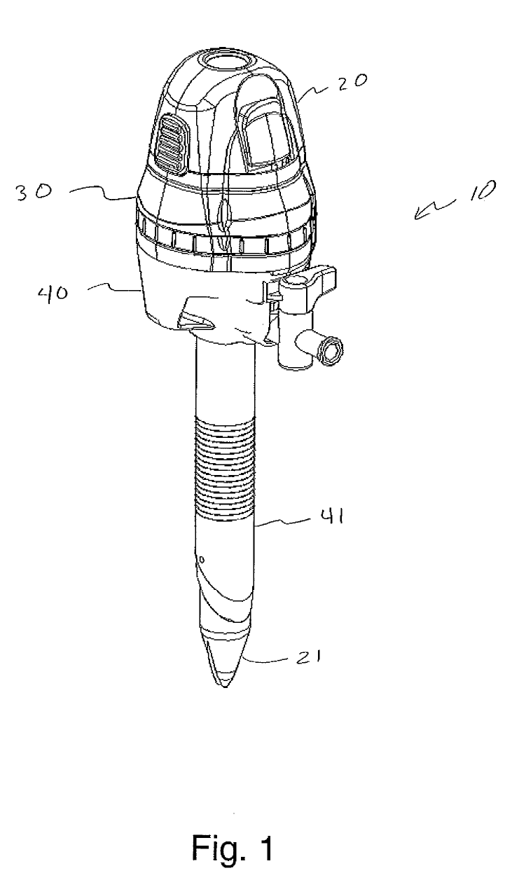

FIG. 1 depicts one embodiment of an access device (10). This example the device is a trocar suitable for abdominal surgery, but other access devices are also contemplated. The access device (10) comprises an upper housing (30) and a lower housing (40). A cannula (41) extends distally from the lower housing (40) and defines a longitudinal axis. The cannula (41) retracts the abdominal wall tissue and provides a port through which surgical instrument may be introduced into the patient. In this embodiment the cannula (41) may be in the form of a rigid tube, but other cannulas known in the art may also be used including flexible or variable length retractors. The access device (10) may optionally include a removable obtruator (20) extending through the cannula (41) with a distal tip (21) adapted to perforate tissue and facilitate insertion of the cannula (41) in the abdominal wall.

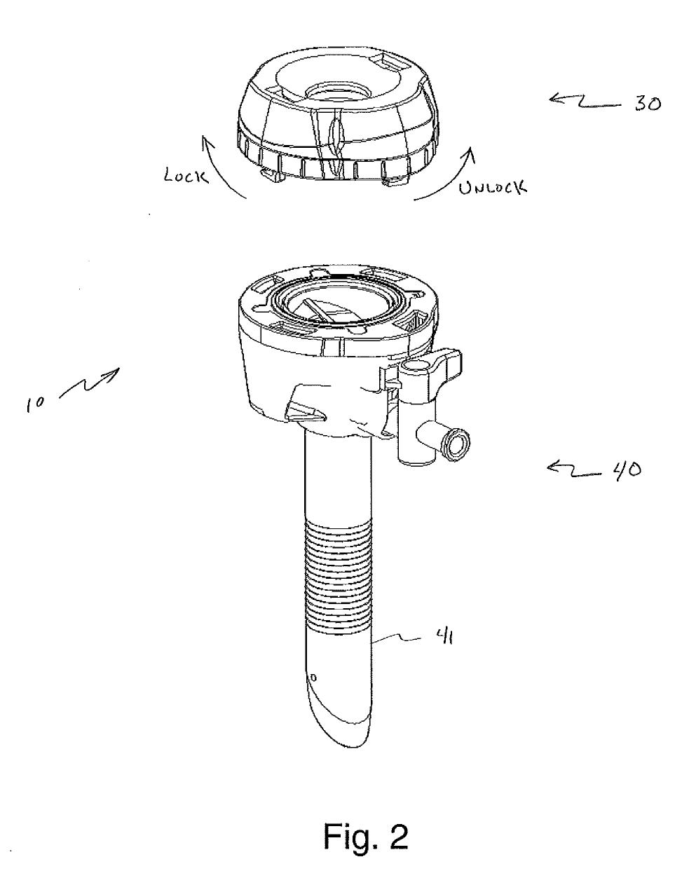

FIG. 2 depicts the upper housing (30) removed from the lower housing (40). The upper housing (30) comprises a proximal face, a distal face, and a bore extending between the proximal and distal faces and aligned with the cannula (41). The upper housing (30) may have instrument seal in the bore adapted to seal against surgical instruments positioned in the access device (10) to prevent insufflatory fluids from escaping. The lower housing (40) may include a zero-closure seal adapted to seal the access device (10) to prevent insufflatory fluids from escaping when an instrument is not positioned in the access device (10).

As one with ordinary skill in the art will recognize, during surgery it may be desirable to remove the upper housing (30), such as when removing larger tissue samples from the patient. The upper housing (30) operates as a removable cap for the access device (10). In this embodiment the upper housing (30) includes a twist-to-lock mechanism such that clock-wise rotation will lock the upper housing (30) to the lower housing (40), and counter clock-wise rotation will unlock the upper housing (30).

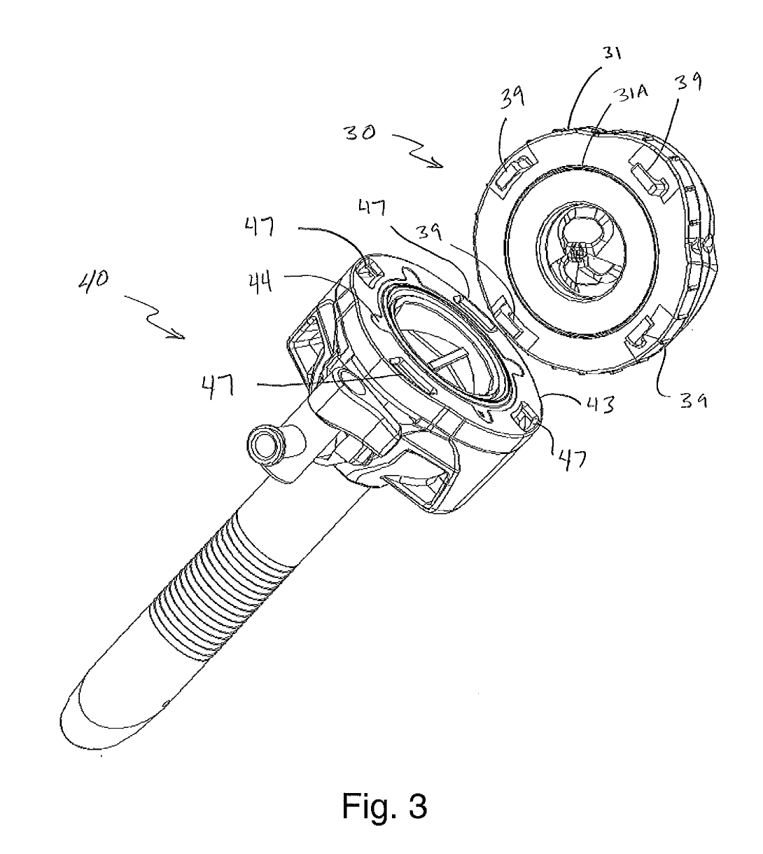

FIG. 3 depicts a detailed view of an attachment mechanism. The upper housing (30) includes a plurality of projections (39) extending from the distal face of the base (31). This embodiment includes four projections (39), but fewer or more may also be used. The projections (39) may each include a transverse tab, which as shown in this example are oriented circumferentially to form a generally L-shaped projection (39). In this embodiment the tabs are oriented counter clock-wise when viewing the distal face, but the tabs could also be oriented clock-wise.

The lower housing (40) includes a plurality of apertures (47) in the proximal face of the housing cap (43). The apertures (47) are dimensioned to receive the distal projections (39). In this embodiment apertures (47) are oblong and oriented circumferentially, but other shapes are also possible. Once the tabs are positioned in the apertures (47), rotation of the upper housing (30) will move the tabs to engage under the housing cap (43) thus locking the upper housing (30) to the lower housing (40). A slight interference or “snap-fit” between the projections (39) and housing cap (43) may be provided to resist inadvertent rotation in the opposite direction thus preventing unintentional release of the upper housing (30).

A resilient seal (44) is provided on the proximal face of the housing cap (43) to prevent insufflatory fluids from escaping between the upper and lower housings. The distal face of the base (31) may include a raised ring (31A) to engage the seal (44).

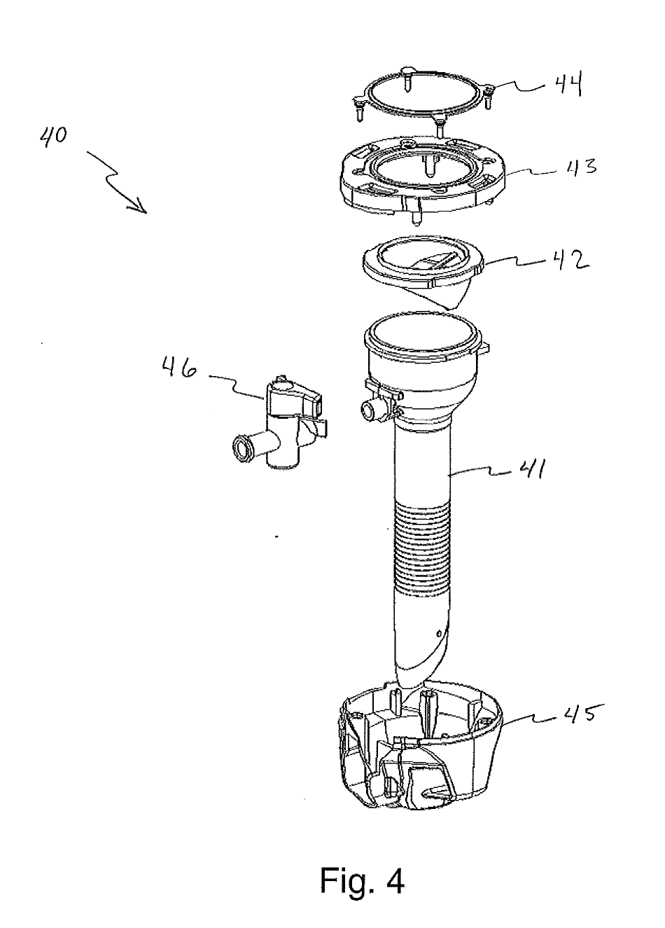

FIG. 4 depicts an exemplary assembly for the lower housing (40). A zero closure seal (42) is nested in the proximal opening of the cannula (41). In this embodiment the zero-closure seal (42) is a “duck-bill” seal, but other types of zero-closure seals known in the art may also be used. The distal end of the cannula (41) is inserted through the housing (45). The housing engages flared proximal end of the cannula (41) and attaches to the housing cap (43), thus sandwiching the zero-closure seal (42) between the proximal surface of the cannula (41) and the distal face of the housing cap (43). A stop cock assembly (46) attaches to the cannula (41) to control insufflation. Seal (44) is attached to the housing cap (43).

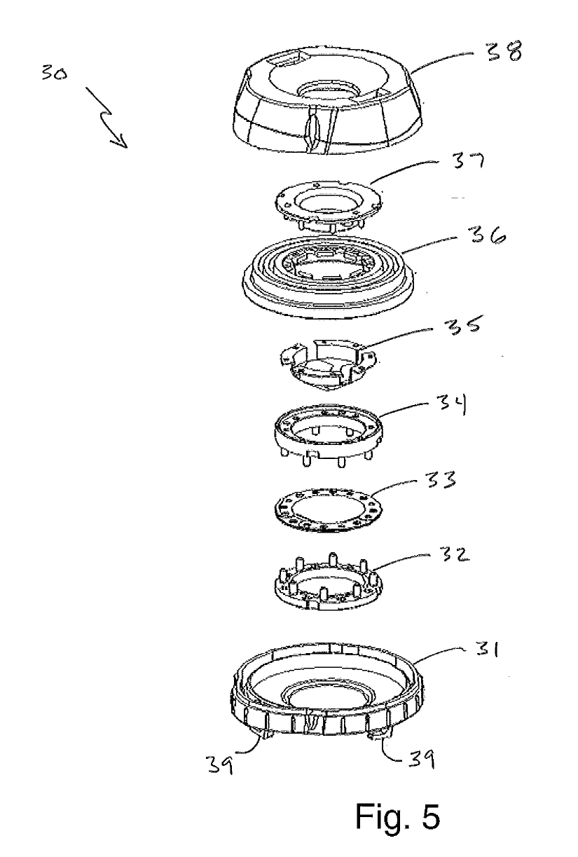

FIG. 5 depicts an exemplary assembly for the upper housing (30). A base (31) engages a upper cap (38) to contain an instrument seal. In the present example the instrument seal includes a lower ring (32), a woven seal (33), a top ring (34), a woven seal protector (35), bellows (36), and a crown (37); however, other types of instrument seals known in the art may also be used.

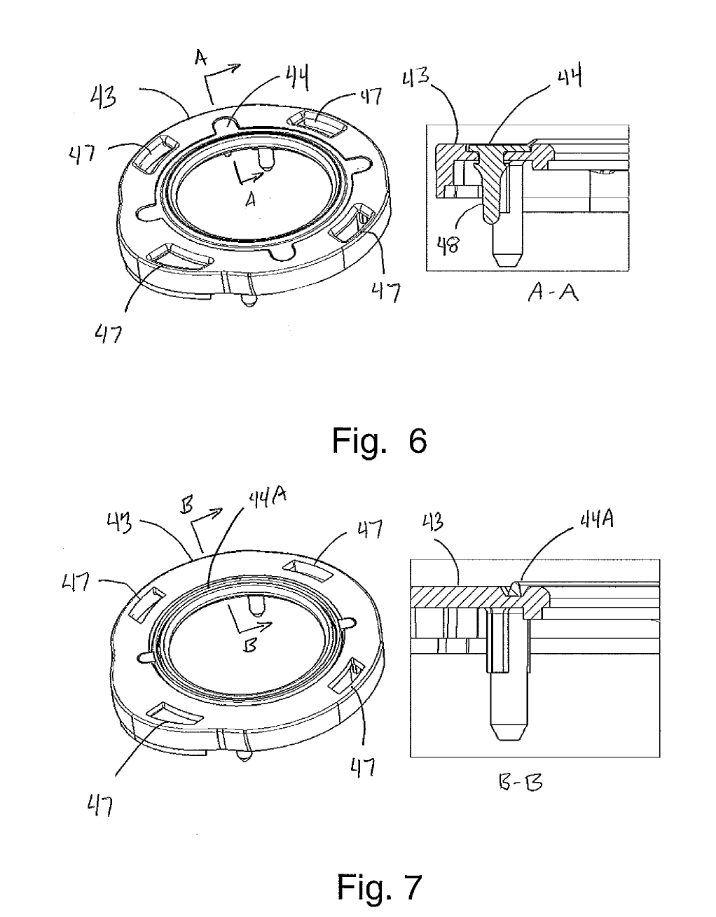

FIG. 6 depicts a detailed view of the proximal face of the housing cap (43). Cross-section A-A illustrates one technique for attaching the seal (44) to the housing cap (43). The seal (44) includes a plurality of distally extending barbed fingers (48) that engage with holes in the housing cap (43). In this example, the seal (44) is recessed in the housing cap (43) with the exposed surface of the seal (44) substantially even with distal face of the housing cap (43).

FIG. 7 depicts an alternative proximal face of a housing cap (43). Cross-section B-B illustrates a technique wherein the seal (44A) is over-molded onto the housing cap (43). In this example, the seal (44A) is raised above the distal face of the housing cap (43).

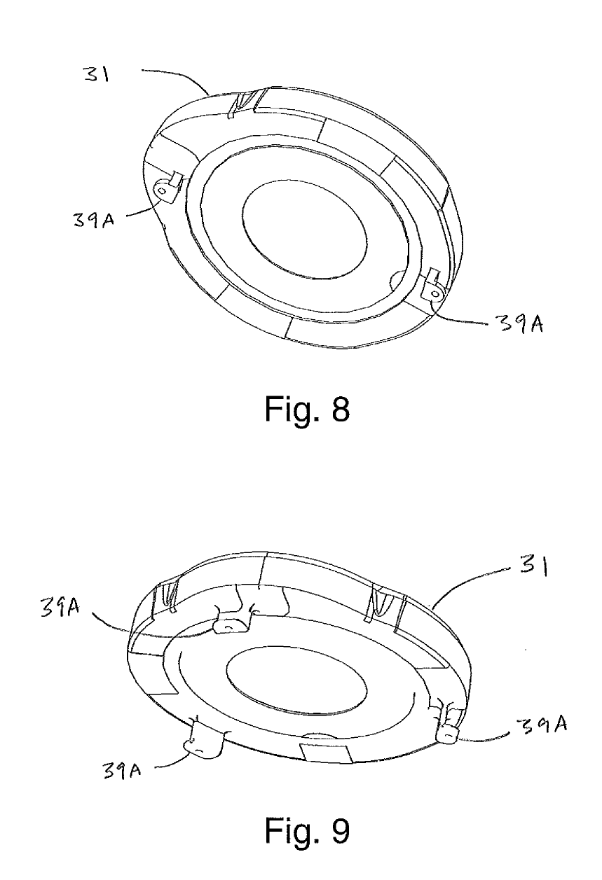

FIG. 8 depicts an alternative embodiment of a base (31) comprising two distal projections (39A) with laterally oriented tabs. The distal projections (39A) may engage appropriate shaped apertures in a housing cap.

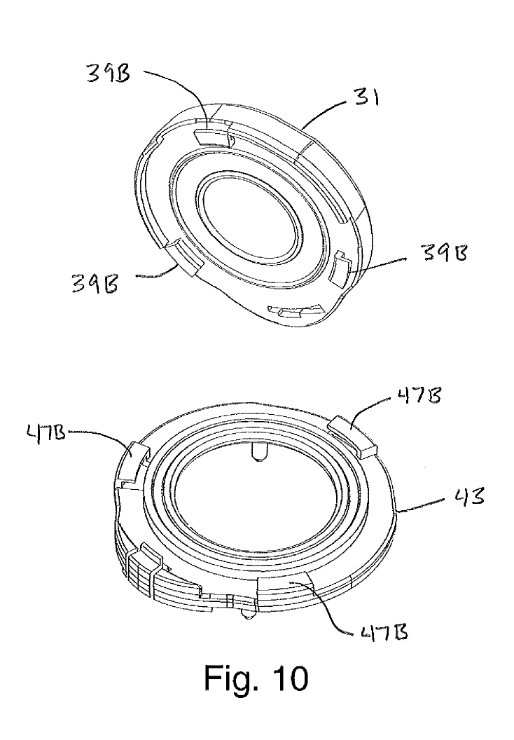

FIG. 9 depicts an alternative embodiment of a base (31) comprising three distal projections (39A) with laterally oriented tabs. The distal projections (39A) may engage appropriate shaped apertures in a housing cap.

FIG. 10 depicts an alternative embodiment of a base (31) and housing cap (43). The base (31) comprises a plurality of distal projections (39B) with laterally oriented tabs. The housing cap (43) comprises a plurality of proximal projections (47B) each comprising a medially oriented tab adapted to mate with the laterally oriented tabs.

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the specific materials, dimensions, and the scale of drawings will be understood to be non-limiting examples. Accordingly, the scope of the present invention should be considered in terms of the following claims and is understood not to be limited to the details of structure, materials, or acts shown and described in the specification and drawings.

1. A trocar, comprising:
a) a cannula having a proximal end and a distal end;
b) a housing connected to the proximal end of the cannula, the housing containing a zero-closure seal;
c) a removable cap the containing an instrument seal, the removable cap comprising a plurality of distal projections providing a twist-to-lock attachment to the housing.

2. The trocar of claim 1, further comprising an obtruator.

3. The trocar of claim 1, wherein the housing comprises housing cap.

4. The trocar of claim 3, wherein the housing cap comprises a seal engaging the removable cap.

5. The trocar of claim 3, wherein the housing cap comprises a plurality of apertures dimensioned to receive the distal projections.

6. The trocar of claim 5, wherein the apertures are oblong and oriented circumferentially.

7. The trocar of claim 1, wherein the distal projections each comprise a circumferentially oriented tab.

8. The trocar of claim 7, wherein the tabs are oriented counter clock-wise.

9. The trocar of claim 7, wherein the distal projections and tabs are generally L-shaped.

10. The trocar of claim 1, wherein the distal projections each comprise a laterally oriented tab.

11. The trocar of claim 10, wherein the housing cap comprises a plurality of proximal projections each comprising a medially oriented tab adapted to mate with the laterally oriented tabs.

12. A removable cap for a surgical access device having a lower portion, the removable cap comprising:
a) an upper housing having a proximal face, a distal face, and a bore extending between the proximal and distal faces;
b) an instrument seal in the bore;
c) a plurality of projections from the distal face adapted mate with the lower portion such that rotation of the upper housing about the bore in a first direction locks the upper housing to the lower portion, and rotation of the upper housing about the bore in a second direction unlock the upper housing from the lower portion.

13. The removable cap of claim 12, wherein the distal projections each comprise a circumferentially oriented tab.

14. The removable cap of claim 13, wherein the tabs are oriented counter clock-wise.

15. The removable cap of claim 12, wherein the distal projections each comprise a laterally oriented tab.

16. The removable cap of claim 12, further comprising a raised ring on the distal face circumscribing the bore and adapted to engage a seal on the lower portion.

17. A trocar, comprising:
a) the removable cap of claim 12;
b) a lower portion comprising a lower housing with a zero-closure seal; and
c) a cannula extending distally from the lower housing.

18. The trocar of claim 17, further comprising an obtruator.

19. A surgical access device, comprising:
a) a cannula having a proximal end and a distal end;
b) a housing connected to the proximal end of the cannula and containing a zero-closure seal, the housing comprising a housing cap with a plurality of oblong apertures;
c) a removable cap containing an instrument seal, the removable cap comprising a plurality of distally extending L-shaped projections having a twist-to-lock engagement with the oblong apertures to attach the removable cap to the housing.

20. The surgical access device of claim 19, wherein the cannula is rigid.
